(19) United States
(12) Patent Application Publication
(10) Pub. No.: US 2021/0283193 A1
(43) Pub. Date: Sep. 16, 2021

(54) Solid honey product and method of producing the same

(71) Applicant: Endre Péter MARTON, Szigethalom (HU)

(72) Inventor: Endre Péter MARTON, Szigethalom (HU)

(21) Appl. No.: 17/120,547

(22) Filed: Dec. 14, 2020

Related U.S. Application Data
(60) Provisional application No. 62/987,415, filed on Mar. 10, 2020.

(51) Int. Cl. A61K 35/644; A61K 31/352; A61K 31/05; A61K 47/26; A61K 36/185; A61K 45/06; A23L 21/25; A23L 33/105; A23G 3/42; A23G 3/48; A23G 3/54; A23L 3/40; A23L 33/15

(52) U.S. Cl. CPC A61K 35/644; A61K 31/352; A61K 31/05; A61K 47/26; A61K 36/185; A61K 45/06; A23V 2002/00; A23L 33/105; A23G 3/42; A23G 3/48; A23G 3/54; A23L 3/40; A23L 33/15; A23L 21/25

(57) ABSTRACT

A method of producing a solid honey product from natural honey is disclosed. The method comprises dehydrating natural honey by heating the honey up to a temperature between 50-70° C. in a closed container under an air pressure of 0.001-0.2 bar, wherein the temperature is selected so that it exceeds the boiling point of the water at the applied air pressure, and by removing the generated steam from the closed container, wherein the step of dehydration is carried out until the moisture content of the honey decreases to 1-5% w/w; shaping the dehydrated honey; and cooling down the shaped honey to ambient temperature and thereby solidifying it. A solid honey product produced by the method consists essentially of honey and having a moisture content within a range of 1-5% w/w.

The present invention relates to a solid honey product and a method of producing the same.

Numerous outstanding effects of honey have been known since the beginning of mankind. Honey is not only nourishment but also an important source of energy for human body. Its effects in domestic medicine have been known for a long time and have been scientifically supported by some recent researches.

In addition to its healing effects, honey is also part of the everyday nourishment due to its excellent taste. It is used as a base ingredient in confectioneries, cakes and foods and also as a sweetening agent.

Unfortunately, even having a number of positive effects, the versatile use of honey is limited by its consistence because at ambient temperature, honey is sticky and slimy, it is difficult to wrap up and to store. That is why honey is typically consumed in a processed form. The solid honey products eliminate the afore-mentioned drawbacks.

Honey can be solidified by removing the moisture from it. Normally, the moisture content of honey that complies with the food industry standards is at most 20% (w/w), which makes honey a liquid and highly viscous material at ambient temperature. Viscosity of honey strongly depends on the type of honey, the time of collection and its moisture content.

The process of solidifying honey and keeping it in a solid state at ambient temperature consists of three main technological steps, namely dehydration, shaping and moisture protection.

Dehydration

Removal of the water from honey cannot be carried out in a natural way because honey is a highly hygroscopic material and thus under normal ambient conditions, the amount of water exiting from honey and the amount of water adsorbed by honey are near the same within a certain time period. Therefore honey is uncapable of releasing all of its moisture content.

One possible way to remove water from honey is to increase the temperature of honey, which facilitates evaporation of the water. However, at a temperature close to the boiling point of water, typically at 90-98° C., which is necessary for the water to evaporate, the sugars, the active ingredients and the enzymes contained in the honey are also damaged, which may significantly deteriorate the quality of the end product.

Furthermore, generation of hydroxymethylfurfural (HMF) during heating and the adverse effect of the heating to the activity of the enzymes of honey are not negligible either.

Laboratory experiments have proven that the undesired amount of HMF measured in a specific honey is inversely proportional to the amount of other desired active ingredients present in honey. During processing it should also be taken into account that the change in the amount of the useful ingredients of honey (e.g. acids, enzymes, etc.) is not a linear function of the processing temperature and the duration of processing.

The production of high-quality solid honey products can be achieved by means of vacuum drying wherein honey is placed into a closed low-pressure chamber, a so-called reaction chamber. By reducing the pressure, the boiling point of the water contained in the honey decreases, so evaporation of the water can be achieved even at a lower temperature. Upon providing a suitably low temperature, the water contained in the honey tends to boil even at a temperature where the active ingredients of the honey are not damaged by the heat. In vacuum the moisture content of honey transits into a gaseous state as a result of boiling and it exits from the honey which, in turn, has a higher boiling point. By the detraction of the vapor, even the entire amount of water can be extracted from the honey, and thus the material remaining after extraction of the water is a solid material at ambient temperature and at normal ambient pressure, it has an amorphous crystal structure and it contains all non-volatile components of honey.

It is important that dehydration should be carried out within the shortest possible time period and at the lowest possible temperature in order to keep the amount of enzymes of honey at the highest possible rate. The ingredients of the honeys collected in different seasons, from different plants and at different locations, in particular the composition of the sugars contained in the honey, may be significantly different. The tendency of crystallization strongly depends on the rate of the glucose to other ingredients, which are mainly sugars. This rate influences the particular moisture content at which the dehydrated honey can spontaneously undergo crystallization at ambient temperature due to its substantially low moisture content. Once crystallization has occurred and the solidified honey product is in a quasi non-sticky form or requires only a minor non-adherence treatment for further processing, it is suggested terminating the process at that specific moisture content, which is anyway characteristic to the time and place of collection of the honey. At such a temperature, the desired solid state can be achieved, and the useful ingredients present in the honey are subject to less adverse effect, resulting in their higher quality and higher amount in the honey product. The moisture content needed for the spontaneous crystallization of a specific honey should be kept within a range of 1-5% (w/w). It is noted, however, that by applying vacuum drying the moisture content can be reduced even to 0% (w/w).

A solid honey product may also contain any additional substances that can enhance the physiological effects, the taste and the value of delight of honey. The solid honey product can, therefore behave even as a kind of carrier or “container”, which means that during production various natural components (e.g. herbs, fruit concentrates, natural flavors, natural colorants) can be added to the honey to provide a product wholly consisting of natural ingredients that does not contain any synthetic additive, improver, gelatinizing agent or preservative for achieving the desired material consistency.

Nevertheless, a solid honey product is capable of receiving synthetic components (e.g. synthetic active ingredients, artificial flavors, artificial colorants) during its production. The synthetic additives can be mixed to the honey typically in the form of a dry powder, an oil or an oil solution, an aqueous solution or an alcoholic solution. The proportion of an additive to the honey is defined uniquely, depending on the characteristics of the specific additive. The amount of an additive can be increased only to a limit value which depends on the characteristics of the particular additive, in particular its state of matter, as well as the effect of the additive on the crystallization of the sugars present in the honey.

When volatile active ingredients are added, it is preferred that after feeding an active ingredient into the reaction chamber, the pressure of the reaction chamber is increased to such a high level that the boiling point of the volatile active ingredient at this pressure level exceeds the temperature of the molten honey present in the reaction chamber.

Shaping

After dehydration, the next step of the production technology is to shape honey that has the desired low moisture content, or contains no water at all, and optionally contains some additives. Various techniques are known for this purpose.

In case of casting, after dehydration, honey is changed into a liquid state by heating it to a suitable temperature so that its viscosity corresponds to a level that allows its casting into a plurality of pre-formed molds, typically made of silicone, by means of a dispensing apparatus (e.g. Piston filler). The dispensed honey cools down in the molds and takes up their shapes while getting solid.

In case of pressing, after dehydration, honey is cooled down to a state in which it is not liquid but yet formable, and then it is pressed into a plurality of pieces of desired size and shape by means of two counter-rotating, patterned cylinders. In this technology it is also conceivable that the dehydrated honey having additives mixed therein is pressed to have a form of a cylindrical rod of a desired size at a suitable temperature by means of an extruder, and then the dehydrated honey rod is cut into pieces that are individually put into a plurality of molds and pressed to have the desired shape. This solution has the advantage of applying a relatively low temperature during both extrusion and pressing, so the shape, the size and the surface pattern of the pressed product may vary in a wide range.

Moisture Protection

Honey is a highly hygroscopic material. After removal of the water this property gets even stronger, and thus during the shaping process, dehydrated honey immediately starts adsorbing the moisture present in the ambient air. It is therefore preferred that the shaped product is covered with a honey-compatible component that assists further processing and allows packaging the shaped product. For a non-adherence treatment of the shaped product, two alternative solutions may be efficiently used: covering with powder and tropicalization.

In case of covering with powder, the shaped products are covered with the powder of a material similar or identical to any one of the components of the honey. Covering may be carried out either during the shaping step, or after the shaping step. Due to the covering, the product will temporarily be powder-dry and thus suitable for nonstick packaging. The covering material may include a powder-like natural sugar (e.g. fructose powder) or the powder of an artificial sugar (e.g. maltodextrin).

In case of tropicalization, the surface of the product is covered with a hydrophobic material such as palm oil or bee wax, which makes the product suitable for packaging. This solution reduces water adsorption capability of the product and thus the surface adherence of the product is substantially reduced.

In view of the above two steps, there is no doubt that because of the highly hygroscopic property of the product, the primary packaging plays a very important role from the point of view of maintaining the product characteristics for a long time. The primary packaging provides multiple functions simultaneously, including:
protecting the product from the ambient moisture,
protecting the product from sticking together,
allowing re-crystallization of the product after melting,
protecting the product from the ambient pollution,
showing the required product information.

Packaging

For packaging hygroscopic products, two packaging techniques are widely used:
Blister packing: the products are placed into seats formed (blistered) in a plastic foil and then the seats of the foil are closed with another plastic or aluminum foil.
Pillow packing: a hose is first formed from a plastic foil, and the products are then placed within the hose with closing the hose in a section-wise manner.

The applicability of the above packaging techniques depends on the vapor barrier properties of the materials used therein.

When the production capacity and the technological environment achieve a desired level, the step of covering the product with a powder or oil/bee wax can be omitted, provided that the moisture content of the manufacturing space can be kept at a low level and after shaping, the product is placed in the moisture-proof packing within the shortest possible time.

A method of producing a solid honey product by vacuum drying is disclosed, for example, in the document US 2014/112997 A1. In this method liquid honey is heated at least to 98° C. under a vacuum pressure of at least 27 inHg (680 mmHg), and said temperature and pressure are maintained until the moisture content of honey decreases below 1% (w/w). Optionally, flavors, pharmaceutical active agents or other auxiliary agents (e.g. anti-stick material) are mixed into the thus obtained dehydrated, molten honey, and then it is shaped into ready-for-use units.

One of the drawbacks of the above method is that as honey is heated to a temperature of at least 98° C., enzyme activity of the honey practically ceases within a short time, typically in a few minutes (see Nikolett Czipa, “Comparative analysis of honeys of different origin and the effect of the production technology to the quality” (Ph.D Thesis, 2010, University of Debrecen, Hungary).

Finally, it is also a problem that after dehydration, the temperature of the molten honey is relatively high (above 90° C.), so certain additives (e.g. vitamin E) can be introduced into the molten honey only through substantial reduction in quality.

It is an object of the present invention to eliminate the above-mentioned drawbacks. The invention is based on the core idea that if liquid honey having a typical moisture content of approximately at most 20% is dehydrated at a temperature of at most 70° C. and at a pressure of approximately at most 0.2 bar (or below), the enzyme activity of the honey will remain at least in part during the process and any flavor, pharmaceutical active agent or other additive may be mixed into the molten honey of lower temperature without significant decrease in quality.

The above objects are achieved by providing a method comprising the steps of:
dehydrating natural honey by heating the honey up to a temperature between 50-70° C. in a closed container under an air pressure of 0.001-0.2 bar, wherein the temperature is selected so that it exceeds the boiling point of the water at the applied air pressure, and by removing the generated steam from the closed container, wherein the step of dehydration is carried out until the moisture content of the honey decreases to 1-5% (w/w);
shaping the dehydrated honey; and
cooling down the shaped honey to ambient temperature and thereby solidifying it.

The above objects are further achieved by providing a solid honey product consisting essentially of honey and having a moisture content within a range of 1-5% (w/w).

The invention will now be described in detail with reference to the drawings, in which

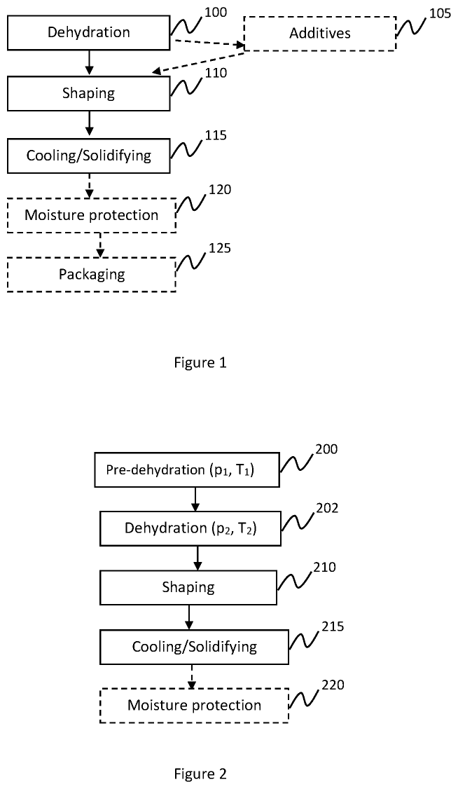

FIG. 1 is a flow diagram illustrating the main steps of the method according to the invention.

FIG. 2 is a flow diagram illustrating the main steps of a preferred embodiment the method according to the invention.

As FIG. 1 shows, in a first step 100 of the method according to the present invention, natural honey is dehydrated. In one embodiment of the method, honey is filled into an air-tightly sealed container, a so-called reaction chamber, in which vacuum can be generated. The inner space of the reaction chamber is heated to at least 50° C., but at most 70° C., while in the reaction chamber a pressure of at least 0.001 bar (0.76 mmHg), but at most 0.2 bar (152 mmHg) is continuously maintained. Since at a pressure of 0.2 bar the boiling point of the water is somewhat lower than 60° C., the temperature of the water is above its boiling point at this specific temperature and pressure, so water tends to boil and evaporate from the honey. By reducing the pressure in the reaction chamber, the temperature may also be reduced. The temperature and the pressure of the reaction chamber is regulated so that at a particular pressure, the temperature be above the boiling point of the water.

In another embodiment of the present invention, the step 100 of dehydration comprises heating the reaction chamber up to the dehydration temperature, i.e. to 50-70° C., and the pressure is set to at most 0.1 bar. Then honey, which has priorly been heated to approximately 40° C., is injected into the reaction chamber through one or more nozzles of small diameter (max. 2 mm). Due to the difference between the ambient pressure and the pressure prevailing in the reaction chamber, the pre-heated honey flows into the reaction chamber through the nozzles. During injection, the pressure of the reaction chamber is to be maintained at a fixed level. Upon termination of the injection process, the nozzles are to be closed. The injected honey may be either pre-treated (e.g. filtered, pasteurized, etc.) or untreated. Due this technique, dehydration may be significantly faster and much more efficient.

During the dehydration process, the steam is removed from the reaction chamber through an evacuation outlet into a condenser arranged between the reaction chamber and a vacuum pump. The steam will then precipitate in the condenser. If the temperature of the lamellas of the condenser is maintained below 3° C., the steam evacuation will be quite efficient. Dehydration is stopped when the moisture content of the honey decreases into a range of 1-5% (w/w).

Increasing the efficiency of dehydration is important in order to protect the honey components. Although it would be a promising option to increase the temperature of the honey above 70° C. in order to accelerate evaporation, this technique would lead to a reduction in the amount of those honey components that substantially contribute to the beneficial effects of honey (e.g. flavonoids, proline, polyphenols, etc.), to the generation of certain undesired substances (e.g. hydroxymethylfurfural) or a significant increase in the amount thereof, and to provide an unfavorable effect to the structure of the sugars present in the honey. Consequently, it is preferred to keep the temperature at the lowest possible level, at a maximum of 70° C., preferably below 60° C. By reducing the temperature along with increasing the vacuum, the process of dehydration may be accelerated. It is preferred to decrease the pressure close to 0.001 bar because in this case it is enough to heat the reaction chamber only to approximately 50° C.

The duration of dehydration may be significantly reduced by increasing the surface-to-volume ratio of the honey placed in the reaction chamber, and also by continuously stirring the heated honey inside the low-pressure reaction chamber. In addition to the evaporation surface and the temperature of the honey, as well as the speed and efficiency of stirring, the particular content of the honey (which depends on the type of the honey, the characteristics of the collection area, the season, the weather, etc.) also influences the speed of dehydration, so it is difficult to provide an exact calculation model for it. The optimal values of the parameters should be determined by one skilled in the art through experiments.

Stirring has a double function. By using a stirring blade with an appropriate shape, the steam bubbles that are generated in the honey during the dehydration process are facilitated to get to the surface of honey and to exit. Furthermore, the stirring blade plays an important role in quickly, efficiently and homogenously introducing the active ingredients into the dehydrated honey at the end of the dehydration process.

The dehydration process can be regarded as finished when the honey in the reaction chamber is ready for further processing.

Once the desired moisture content of 1-5% (w/w) has been reached, the dehydrated honey produced in the reaction chamber is shaped in a next step 110. Shaping may be carried out by the above-mentioned casting or pressing process.

In case of casting, the dehydrated but yet plastic honey is cast into molds, and in step 115, it is cooled down to ambient temperature (ca. 20-22° C.). In the step of cooling, honey is preferably allowed to cool down by itself. At the above specified moisture content, the dehydrated honey transits into a material that is solid and crystal at ambient pressure and temperature.

In case of pressing, the dehydrated plastic honey is pressed into the desired shape by means of rollers, and in step 115, like after casting, it is cooled down to ambient temperature (ca. 20-22° C.).

In a further optional step 120, the shaped solid honey products are preferably provided with moisture protection (e.g. covering with sugar powder or tropicalization), and upon demand, in step 125, they may be packaged according to the intended use (e.g. blister packing, pillow packing).

The speed of the above introduced manufacturing process may be further increased by performing the dehydration process continuously, instead of performing it intermittently. In the intermittent mode, the dehydration process is to be stopped periodically because of the need of discharging the reactor (i.e. drawing off the dehydrated honey), so the processing of the molten honey cannot be continuous. This problem is eliminated by a preferred embodiment of the method of the invention, wherein the dehydration process is carried out in two phases. The main steps of this embodiment of the method are illustrated in FIG. 2.

In a first step 200, the natural honey is introduced into a first reaction chamber, (i.e. a closed container), where in a first phase, pre-dehydration is performed for reducing the moisture content of honey to a pre-determined intermediate value, typically to 5-10% (w/w) at a first vacuum pressure p₁ and a first temperature T₁. The honey of reduced moisture content is then fed from the first reaction chamber into a second reaction chamber (i.e. a closed container) where in a step 202, the dehydration process is continued at a second vacuum pressure p₂ and a second temperature T₂, which are different from the first pressure p₁ and the first temperature T₁, respectively, until the final desired moisture content of 1-5% (w/w) is reached. Since the dehydration process is not linear and different external conditions (pressure, temperature, evaporation surface, stirring speed, etc.) are optimal along different sections of the dehydration curve, dehydration can be further accelerated due to this two-phase process while preserving the useful enzymes present in the honey. By appropriate dimensioning of the reaction chambers and optionally interposing a buffer unit therebetween, feeding the base material (i.e. natural honey) into the system and drawing off the dehydrated honey having the desired moisture content may be performed continuously, thereby providing an optimal speed for the subsequent shaping and packaging processes.

After completing the above described two-phase dehydration process, the method proceeds in a known way, that is, in step 210 the dehydrated honey is shaped, and in step 215 the shaped honey is cooled down and solidified. If necessary, in a further step 220, the shaped and solid honey product may be provided with moisture protection.

After dehydration, the dehydrated honey becomes a material with a somewhat higher melting point, which is typically 40° C. The melting point of the dehydrated honey strongly depends on the components of the honey. Accordingly, at a higher temperature the molten honey is suitable for adding other components to the dehydrated honey.

In a preferred embodiment of the method according to the present invention, after the step of dehydration, in a further step 105, one or more materials selected from the following group may, for example, be added to the molten honey once the vacuum is eliminated in the reaction chamber: natural or artificial flavors, colorants, vitamins, nutritional supplements, herbal extracts, pharmaceutical active agents, coffee or tea extracts, fungus extracts, etc. The additives may include any kind of natural or artificial materials that do not block crystallization of the molten honey at lower temperatures. The additives may include water-soluble, alcohol-soluble or oil-soluble, natural or artificial components, and they may be in the form of dry powder, mill product or liquid. Since after dehydration viscosity of the dehydrated honey is very high, it is also possible to add an additive that can exert a desired effect, during permanent stirring, in the form of homogenous particles having small droplet size or small grain size.

Due to its unique characteristics, honey is particularly suitable for administering certain active agents into the human body. Recently, rapid development has been experienced about the use of extracts made from cannabis (Cannabaceae), in particular, the species of the genus Cannabis (Cannabis sativa, Cannabis indica, Cannabis ruderalis, Cannabis spontanea) and the species of the genus Humulus, as well as certain active agents extracted therefrom and their respective synthetic variants. A high demand has arisen for administering cannabidiol (CBD) or tetrahydrocannabinol (THC) using a honey product. These extracts and active agents are currently consumed mostly in the form of oils, which has a number of drawbacks. First of all, the transportation of such products (typically in small bottles), the dispensing thereof (typically by means of a pipette) and their administration into a patient's mouth (typically through dropping into the mouth using a pipette) are inconvenient and unprecise, thereby limiting the consumability of these materials. Administering the afore-mentioned active agents in a honey pastille allows precise dosing and, as a further advantage, the taste of the honey and the natural flavors mixed to the honey (e.g. herbs, of fruit juice concentrates) can compensate the often unpleasant taste of such pharmaceutical extracts in many cases, or they may assist to achieve a rapid and intensive absorption of the active agents, even with multiple efficiency with respect to the active agents solved in oil. The honey pastille is also suitable for administering active agents that are dissolvable in a number of other materials (e.g. oil, water, ethyl-alcohol, etc.). Due to the method according to the invention, the above-mentioned active agents can be added to the honey product in a way that the production loss can be kept at a low level while the amount of the active agents added to the honey product hardly changes.

After mixing the pre-determined components, the molten honey containing the additives is shaped in way as described above, and the step of moisture protection and the step of packaging are carried out upon demand.

In case of continuous production, mixing the active agents to the honey is preferably to be carried out after the dehydration process, in a special purpose, intermediary bowl. This allows for the honey to be subject to dehydration without containing additives, and the active agents can be added, before the shaping step, in the desired concentration to the dehydrated honey of appropriate amount, without disrupting the dehydration process.

The method according to the present invention allows the production of a solid honey product that has a moisture content of 1-5% (w/w). The honey product may consist only of honey (100% honey content), or it may contain additives as well. The one or more additives may include natural or artificial flavors, colorants, vitamins, nutritional supplements, herbal extracts or pharmaceutical active agents. It is particularly preferred that the solid honey product contains an extract of Cannabis, for example a composition containing cannabidiol (CBD) or tetrahydro-cannabinol (THC).

The solid honey product according to the present invention may be used in many fields and ways that have not been available so far, including without limitation:
the honey product in itself (without flavoring) as a natural confectionery (candy)
flavored confectionery (flavored, for example, by fruit concentrates, herbs or spices, coffee extract, tea extract, etc.)
nutritional supplements with vitamins or herbs
sugar substitutes as natural sweeteners
confectionary base ingredients that have not been known or used before
base ingredients of sweets industry that have not been known or used before
a substance used to introduce a pharmaceutical active agent into the body.

The solid honey products made by the method according to the invention may be produced from the dehydrated honey in numerous various forms, such as
pastille
hard candy
lollipop
coarse-grained mill product
fine-grained mill product
product with rod
candy-like product

One of the most important advantages of the method according to the invention is that during production, enzyme activity of the honey decreases only to a limited extent, but not entirely, unlike the prior art production technologies wherein due to the heating above 90 C, enzyme activity of the honey decreases to such a low level that is practically zero (see Nikolett Czipa, “Comparative analysis of honeys of different origin and the effect of the production technology to the quality” (Ph.D Thesis, 2010, University of Debrecen, Hungary).

Due to the method according to the invention, an oil-soluble flavor or active agent can be introduced into the dehydrated honey even in a concentration of 5-20% (w/w) so that crystallization will take place, that is, the dehydrated honey product will be of solid state. The specific upper limit of concentration is determined by the components of the actually used honey and the effect of the oil-solved active agents to the crystallization. These parameters can be determined uniquely, in a way specific to the given product.

When other active agents are also added to the solid honey product and those are mixed into the dehydrated honey of relative low temperature (ca. 40-50 C) after the dehydration process, damaging of the added active agents may be minimized.

1. A method of producing a solid honey product from natural honey, the method comprising:
dehydrating natural honey by heating the honey up to a temperature between 50-70° C. in a closed container under an air pressure of 0.001-0.2 bar, wherein the temperature is selected so that it exceeds the boiling point of the water at the applied air pressure, and by removing the generated steam from the closed container, wherein the step of dehydration is carried out until the moisture content of the honey decreases to 1-5% w/w;
shaping the dehydrated honey; and
cooling down the shaped honey to ambient temperature and thereby solidifying it.

2. The method according to claim 1, wherein in the step of dehydration, temperature is maintained below 60° C.

3. The method according to claim 1, wherein the step of dehydration is carried out at a temperature of ca. 50° C. and at a pressure of ca. 0.001-0.01 bar.

4. The method according to claim 1, wherein in the step of dehydration further comprises continuously stirring honey in the closed container.

5. The method according to claim 1, wherein the step of dehydration is carried out in two phases, wherein
in a first phase, reducing the moisture content of honey to 5-10% w/w in a first container at a first vacuum pressure and a first temperature,
feeding the honey of reduced moisture content from the first container into a second container; and
in a second phase, further reducing the moisture content of the honey to 1-5% w/w in the second container at a second vacuum pressure and a second temperature.

6. The method according to claim 1, wherein after the step of dehydration, the method further comprising:
adding one or more additives to the dehydrated honey, wherein the additive has a solid state or liquid state and is in the form of powder, mill product, or a material solved in water, alcohol or oil.

7. The method according to claim 6, wherein the additive is selected from the group of natural or artificial flavor, colorant, vitamin, nutritional supplement, herbal extract, pharmaceutical active agent, other plant derivative or plant extract.

8. The method according to claim 7, wherein additive is an extract of a plant belonging to the family of cannabis (Cannabaceae) and any genus thereof, in particular a plant belonging to the genus Cannabis and Humulus, more particularly a composition containing cannabidiol (CBD) or tetrahydro-cannabinol (THC).

9. The method according to claim 1, further comprising a step of providing the solidified and shaped honey with moisture protection, wherein the solid shaped honey is covered with a sugar powder or tropicalized.

10. The method according to claim 1, further comprising a step of packaging the solid shaped honey, preferably in blister pack or pillow pack.

11. A solid honey product consisting essentially of honey and having a moisture content within a range of 1-5% w/w.

12. The solid honey product according to claim 11, further comprising one or more additives selected from the group of natural or artificial flavor, colorant, vitamin, nutritional supplement, herbal extract and pharmaceutical active agent.

13. The solid honey product according to claim 12, wherein additive is an extract of a plant belonging to the family of cannabis (Cannabaceae) and any genus thereof, in particular a plant belonging to the genus Cannabis and Humulus, more particularly a composition containing cannabidiol (CBD) or tetrahydro-cannabinol (THC).
